(19) United States
(12) Patent Application Publication
(10) Pub. No.: US 2015/0150159 A1
(43) Pub. Date: May 28, 2015

(54) METHOD FOR CONTROLLING FUNGAL DISEASES IN MUSHROOM PRODUCTION

(71) Applicant: DSM IP ASSETS B.V., Heerlen (NL)

(72) Inventor: Jacobus Stark, Echt (NL)

(21) Appl. No.: 14/414,124

(22) PCT Filed: Jul. 16, 2013
(86) PCT No.: PCT/EP2013/065026

§ 371 Date: Jan. 12, 2015

(30) Foreign Application Priority Data
Jul. 17, 2012 (EP) 12176745.3

(51) Int. Cl. A01N 43/90; A01N 43/78; A01G 1/04; A01H 15/00

(52) U.S. Cl. CPC A01N 43/90; A01H 15/00; A01N 43/78; A01G 1/04
USPC 800/297; 514/31; 47/1.1

(57) ABSTRACT

The present invention relates to new antifungal compositions and their use in the method for controlling fungal diseases in mushrooms.

### FIELD OF THE INVENTION

The present invention discloses new antimicrobial compositions to control fungal diseases in the production of mushrooms.

### BACKGROUND OF THE INVENTION

Currently, over twenty mushroom species are commercially cultivated and mushrooms are cultivated in over 60 countries with China, the United States, Poland, the Netherlands and France being the top producers.

The mushroom industry has undergone many changes in the past 10-15 years. Small inefficient farms have closed or merged into larger, more productive farms with increased mechanization and a centralized management. Within this framework, it is essential that fungal disease outbreaks are controlled. Failure to control fungal disease outbreaks in the early stages can be costly, as untreated areas of disease produce spores and propagules that will spread the disease throughout the rest of the farms, leading to a severe reduction in yield and productivity.

The mushroom industry faces major challenges in the 21^{st} century. First of all, fewer fungicides are available to control disease outbreaks, as many fungicides are no longer approved for use. Secondly, there is an increasing demand from consumers to reduce the use of fungicides. Thirdly, due the prolonged and frequent use of fungicides, mushroom pathogens such as Verticillium and Trichoderma have developed resistance to many fungicides (see Grogan, 2008; Romaine et al., 2005; Gea et al., 1997; Romaine et al., 2008).

For many decades, the polyene macrolide antimycotic natamycin has been used to prevent fungal growth on food products such as cheeses and sausages. This natural preservative, which is produced by fermentation using Streptomyces natalensis, is widely used throughout the world as a food preservative and has a long history of safe use in the food industry. It is very effective against all known food spoilage fungi. Although natamycin has been applied for many years in e.g. the cheese industry, up to now development of resistant fungal species has never been observed.

Consequently, it can be concluded that there is a severe need for new and more effective antimicrobial compositions, e.g. antifungal compositions, for the control of fungal diseases in the production of mushrooms.

### DESCRIPTION OF THE INVENTION

The present invention solves the problem by providing a new synergistic antimicrobial, e.g. antifungal, combination comprising natamycin and thiabendazole.

Thiabendazole (4-(1H-1,3-benzodiazol-2-yl)-1,3-thiazole) is broad spectrum systemic fungicide. Examples of commercial products containing thiabendazole are products with the brand names Mintezol®, Tresaderm® and Arbotect®. Said commercial products can be incorporated in the present invention.

It is to be understood that derivatives of natamycin including, but not limited to, salts or solvates of natamycin or modified forms of natamycin may also be applied in the present invention. Examples of commercial products containing natamycin are the products with the brand name Zivion™, like Zivion™ M. Such products are produced by DSM Food Specialties (The Netherlands). Said commercial products can be incorporated in the present invention.

As used herein, the term “synergistic” means that the combined effect of the antifungal compounds when used in combination is greater than their additive effects when used individually.

In general, synergistic activity of two active ingredients can be tested in for example the analysis of variance model using the treatment interaction stratum (see

Slinker, 1998). Relative efficacy can be calculated by means of the following formula: ((value of evolution status of untreated control−value of evolution status of composition)/(value of evolution status of untreated control))*100. An interaction coefficient can then be calculated by means of the following formula: ((relative efficacy of combination compound A+compound B)/(relative efficacy of compound A+relative efficacy of compound B))*100. An interaction coefficient larger than 100 indicates synergy between the compounds.

Alternatively, synergy can be calculated as follows: the antifungal activity (in %) of the individual active ingredients can be determined by calculating the reduction in mould growth observed on products treated with the active ingredients in comparison to the mould growth on products treated with a control composition. The expected antifungal activity (E in %) of the combined antifungal composition comprising both active ingredients can be calculated according to the Colby equation (Colby, 1967):

E=X+Y−[(X·Y)/100], wherein X and Y are the observed antifungal activities (in %) of the individual active ingredients X and Y, respectively. If the observed antifungal activity (O in %) of the combination exceeds the expected antifungal activity (E in %) of the combination and the synergy factor O/E is thus >1.0, the combined application of the active ingredients leads to a synergistic antifungal effect.

In an aspect the invention relates to a method for controlling a fungal disease during the production of mushrooms by applying natamycin and thiabendazole to a substrate wherein mushrooms are growing or are to be grown. Natamycin and thiabendazole are applied in an effective fungal-disease inhibiting amount. In addition, other antifungal and/or antimicrobial compounds can be applied to the substrate either prior to, concomitant with or after treatment of the substrate with natamycin and thiabendazole.

Natamycin and thiabendazole may be applied sequentially to the substrate. The compounds may be applied in any order (first natamycin and then thiabendazole or first thiabendazole and then natamycin). Alternatively, natamycin and thiabendazole may be applied simultaneously to the substrate. In case of simultaneous application, the compounds can be present in different compositions that are applied simultaneously or the compounds may be present in a single composition. In yet another embodiment the antifungal compounds may be applied to the substrate by separate or alternate modes of application.

By applying the compounds, fungal growth on or in the substrate can be prevented. In other words, the compounds protect mushrooms from fungal growth and/or from fungal infection and/or from fungal spoilage. The compounds can also be applied to substrate and/or mushrooms that have been infected with a fungus. By applying the compounds the disease development due to fungi on or in the substrate and/or the mushrooms can be slowed down, stopped or the substrate and/or the mushrooms may even be cured from the disease. Depending on the type of application, the amount of natamycin applied may vary from 5 ppm to 10,000 ppm, preferably from 10 ppm to 5,000 ppm and most preferably from 20 to 1,000 ppm. Depending on the type of application, the amount of thiabendazole applied may vary from 1 ppm to 5,000 ppm, preferably from 5 ppm to 3,000 ppm and most preferably from 10 to 1,000 ppm.

When natamycin is applied in the form of a composition, the composition generally comprises from about 0.005 g/l to about 100 g/l and preferably from about 0.01 g/l to about 50 g/l natamycin. Preferably, the amount is from 0.01 g/l to 3 g/l.

When thiabendazole is applied in the form of a composition, the composition generally comprises from about 0.0001 g/l to about 2000 g/l and preferably from about 0.0005 g/l to about 1500 g/l thiabendazole. More preferably, the amount is from 0.001 g/l to 1000 g/l.

In an embodiment a composition comprising natamycin and/or thiabendazole may further comprise at least one additional compound selected from the group consisting of a sticking agent, a carrier, a colouring agent, a protective colloid, an adhesive, a herbicide, a fertilizer, a thickening agent, a sequestering agent, a thixotropic agent, a surfactant, a further antimicrobial compound, a detergent, a preservative, a spreading agent, a filler, a spray oil, a flow additive, a mineral substance, a solvent, a dispersant, an emulsifier, a wetting agent, a stabiliser, an antifoaming agent, a buffering agent, an UV-absorber and an antioxidant. A further antimicrobial antifungal compound may be an antifungal compound or a compound to combat insects, nematodes, mites and/or bacteria. Of course, the compositions may also comprise two or more of any of the above additional compounds.

The compositions may have a pH of from 1 to 10, preferably of from 2 to 9, more preferably of from 3 to 8 and most preferably of from 4 to 7.

The compositions may be solid, e.g. powders, granulates or tablets. Solid compositions can be used to prepare liquid compositions.

The compositions may also be liquid. The compositions can be aqueous or non-aqueous ready-to-use compositions, but may also be aqueous or non-aqueous concentrated compositions/suspensions or stock compositions, suspensions and/or solutions which before use have to be diluted with a suitable diluent such as water or a buffer system.

Natamycin and thiabendazole may also be applied in the form of a kit. Natamycin and thiabendazole may be present in two separate packages, e.g. containers. The components of the kit may be either in dry form or liquid form in the package. If necessary, the kit may comprise instructions for dissolving or diluting the compounds. In addition, the kit may contain instructions for applying the compounds during the mushroom production process.

As described above, natamycin and thiabendazole are applied to control a fungal disease in mushrooms. The fungal disease can be any diseases in mushrooms caused by a fungus. In an embodiment the fungal disease is caused by a Dactylium species (disease called cobweb or mildew disease), a Diehlomyces species (disease called calves brains or false truffle disease), a Fusarium species (disease called damping off), a Papulaspora species (disease called brown plaster mould disease), a Scopulariopsis species (disease called white plaster mould disease), a Verticillium species (disease called dry bubble disease or brown spot disease), a Mycogone species (disease called wet bubble disease or white mould disease) or a Trichoderma species (disease called green mould disease). In a preferred embodiment the fungal disease is caused by a Verticillium species, a Mycogone species or a Trichoderma species. Even more preferred, the fungal disease is caused by Verticillium fungicola, Mycogone pemiciosa or Trichoderma harzianum, with Verticillium fungicola and Trichoderma harzianum being most preferred.

In an aspect the invention thus relates to a method for inhibiting green mould disease caused by Trichoderma harzianum in mushrooms by applying natamycin and thiabendazole to a substrate wherein mushrooms are growing or are to be grown. In another aspect the invention relates to a method for inhibiting dry bubble disease caused by Verticillium fungicola in mushrooms by applying natamycin and thiabendazole to a substrate wherein mushrooms are growing or are to be grown.

In general, mushroom production can be divided into six steps, phase 1 composting, phase 2 composting, spawning, casing, pinning and cropping. These six steps take approximately 15 weeks to complete.

In the first step (i.e. phase 1 composting), compost is prepared. Compost provides nutrients (e.g. nitrogen and carbohydrate) needed for mushrooms to grow and is thus the substrate wherein mushrooms are growing or are to be grown. Common bulk materials that can be used as compost include wood chips or sawdust, mulched hay, straw-bedded poultry manure, Brewer's grain, waste or recycled paper, coffee pulp or grounds, nut and seed hulls, soybean meal, cottonseed hulls or meal and cocoa bean hulls.

Two types of material are generally used for mushroom compost, the most used and least expensive being wheat straw-bedded horse manure. Synthetic compost is usually made from hay and crushed corncobs, although the term often refers to any mushroom compost where the prime ingredient is not horse manure. Both types of compost require the addition of nitrogen supplements and a conditioning agent, gypsum.

The composting is initiated by mixing and wetting the materials, where after aerobic fermentation commences and eventually compost is made. Phase 1 composting usually takes 7 to 14 days.

The second step is phase 2 composting. This step usually takes 7-18 days. In this step, the compost is finished, meaning ammonia formed during phase 1 composting is removed and the compost is sterilized to kill any insects, nematodes, fungi or other pests that may be present in the compost. Sterilization generally takes place through high or low temperature pasteurization.

How phase 2 composting takes place depends on the type of mushroom production process used. With a bed or shelf system, the compost is placed directly in the beds, which are in the room used for all steps of the mushroom production process.

For the zoned system of growing, compost is packed into wooden trays, the trays are stacked six to eight high, and are moved into an environmentally controlled phase 2 composting room. Thereafter, the trays are moved to special rooms, each designed to provide the optimum environment for each step of the mushroom production process.

The most recently introduced system, the bulk system, is one in which the compost is placed in a cement block bin with a perforated floor and no cover on top of the compost; this is a room specifically designed for phase 2 composting.

The compost, whether placed in beds, trays, or bulk, should be filled uniformly in depth and density or compression.

The third step is spawning. In this step mushroom substrate (i.e. compost) is inoculated with mushroom spawn. Mushroom spawn can be purchased from commercial spawn producers that vegetatively propagate mycelium. The spawn is applied onto the substrate and the obtained substrate is mixed thoroughly. Mixing can be done manually or by means of suitable mixing equipment. If desired, supplements can be added to the substrate. These supplements comprise nutrients and might increase the mushroom yield. Next, optimal conditions for growth of the mycelium through the substrate are chosen. These conditions depend on the substrate dimensions, substrate composition, type of mushroom cultivar, to name just a few. When the mycelium has propagated through the entire substrate layer, the spawning is finished and the next step in the production of mushrooms can be started. The spawning step usually takes 14-21 days.

It is becoming common practice in many countries to prepare fully colonized substrate (i.e. compost) in bulk. This is done in large tunnels. Fully colonized means that the substrate has been subjected to spawning before it is being sold. This is the so-called phase 3 composting. Specialist phase 3 producers sell substrate, eliminating the need for a farm to have its own substrate producing facilities.

The fourth step is casing. In this step a casing layer is applied onto the surface of the substrate. In the casing layer the mushrooms eventually form. Preferably, the casing material is pasteurized to eliminate insects and pathogens. If desired, supplements can be added at casing. These supplements comprise nutrients and might increase the mushroom yield. Preferably, the casing layer is distributed, so the depth is uniform over the surface of the substrate. Such uniformity allows the spawn to move into and through the casing layer at the same rate and, ultimately, for mushrooms to develop at the same time. Casing should be able to hold moisture, since moisture is essential for the development of a firm mushroom. Frequent watering is therefore advised. The casing layer does not necessarily need nutrients. The casing step usually takes 13-20 days.

The fifth step is pinning. In this step the earliest formation of recognizable mushrooms from mycelium takes place. The pins continue to expand to enlarge into mature mushrooms. By adjusting temperature, humidity and carbon dioxide content, the number of pins and the final mushroom size can be controlled. Harvestable mushrooms appear 18 to 21 days after casing.

The sixth and final step is called cropping. It refers to repeating 3- to 5-day harvest periods during the cropping cycle (7 to 10 days). The harvest periods are followed by a few days wherein no mushrooms are available to harvest. The cropping cycle repeats itself in a rhythmic fashion, and harvesting can go on as long as mushrooms continue to mature. Most mushroom farmers harvest for 35 to 42 days, although some harvest a crop for 60 days, and harvest can go on for as long as 150 days. Again, temperature, humidity, and carbon dioxide content are pivotal for optimal productivity.

Freshly harvested mushrooms must be kept refrigerated. To prolong the shelf-life of mushrooms, it is important that mushrooms “breathe” after harvest, so storage in a non-waxed paper bag is preferred to a plastic bag.

After the last mushrooms have been harvested, the growing room should be closed off and the room pasteurized with steam. This final pasteurization is designed to destroy any pests which may be present in the crop or the woodwork in the growing room, thus minimizing the likelihood of infesting the next crop.

Mushrooms can be produced outside in stacks or piles. The sterilization step is then not needed. Since outdoor production is unpredictable and seasonal, less than 5% of commercially sold mushrooms are produced this way.

Preferably, the mushrooms are produced indoors. Indoor growing allows consistent production, regulated by spawning cycles, tight control over growing conditions and substrate composition. This is typically accomplished by windowless, purpose-built buildings, for large-scale commercial mushroom production. Alternatively, mushrooms can also be produced inside caves.

In an embodiment of the present invention the mushrooms are edible. Commercially produced edible mushrooms include, but are not limited to, mushroom species such as Agaricus sp. (such as Agaricus bisporus, Agaricus brunnescens), Auricularia polytricha, Auricularia auricula-judae, Flammulina velutipes, Hypsizygus tessulatus, Lentinus edodes, Pleurotus cornucopiae, Pleurotus eryngii, Pleurotus ostreatus, Rhizopus oligosporus, Sparassis crispa, Tremella fuciformis, Tuber aestivum, Tuber magnatum, Tuber melanosporum, Terfezia ,sp., Ustilago maydis, Coprinus comatus, Morchella esculenta, and Volvariella volvacea.

Natamycin and thiabendazole can be applied during any of the above-mentioned steps of the mushroom production process. They can be applied as pure components or in the presence of a carrier. If desired, each compound can be applied at a different step of the production process, e.g. natamycin can be applied after the casing step, while thiabendazole can be applied after a harvest step. Any combination is possible.

In an embodiment of the method according to the present invention natamycin and thiabendazole are applied to the substrate after spawning.

In another embodiment of the method according to the present invention natamycin and thiabendazole are applied to the substrate after casing. Application can be done directly after the casing layer has been applied. In yet another embodiment of the method according to the present invention natamycin and thiabendazole are applied more than once during the production of mushrooms. For instance, natamycin and thiabendazole can be applied directly after the casing layer has been applied and thereafter once a day for 4 to 5 days. Preferably, natamycin and thiabendazole are applied together with the repeated watering steps that are performed to increase the moisture content of the casing layer. Natamycin and thiabendazole can also be applied during pinning. Moreover, natamycin and thiabendazole can be applied after each harvest of mushrooms.

In an embodiment of the method according to the present invention natamycin and thiabendazole are applied by spraying. Other methods suitable for applying these compounds in liquid form are also a part of the present invention. These include, but are not limited to, dipping, watering, drenching, vaporizing, fogging, fumigating. Spraying applications using automatic systems are known to reduce the labour costs and are cost-effective. Methods and equipment well-known to a person skilled in the art can be used for that purpose.

Natamycin and/or thiabendazole should be used in an effective amount to control a fungal disease in mushrooms. In an embodiment natamycin is applied to the upper surface of the substrate in an amount from 0.01-20 fl. oz. per 1000 sq. ft. (fluid ounces per 1000 square feet), preferably 0.05-10 fl. oz. per 1000 sq. ft., and in particular 0.1-5 fl. oz. per 1000 sq. ft. In an embodiment a composition comprising 1-15 wt % natamycin, preferably 3-14 wt % natamycin, more preferably 5-13 wt % natamycin, and in particular 7-12 wt % natamycin can be applied in the above-mentioned amounts to the upper surface of the substrate. In another embodiment natamycin is applied to the upper surface of the substrate in an amount from 0.1-500 g per 100 m², preferably 1-450 g per 100 m², more preferably 5-400 g per 100 m² and in particular 10-300 g per 100 m². It is well known to a person skilled in the art that application volumes may differ depending on the concentration of natamycin in the compositions applied, Usually, diluted natamycin compositions are applied in a higher volume per surface area unit than concentrated natamycin compositions. It is well within the reach of the skilled artisan to calculate the effective amount of natamycin that needs to be applied to a certain surface area. The natamycin used in the invention is commercialised as a composition comprising 10 wt % natamycin. It is advised to apply 3.1-6.3 fl. oz. per 1000 sq. ft. of this natamycin composition to the upper surface of the substrate.

In an embodiment thiabendazole is applied to the upper surface of the substrate in an amount from 0.01-50 fl. oz. per 1000 sq. ft., preferably 0.05-40 fl. oz. per 1000 sq. ft., and in particular 0.1-30 fl. oz. per 1000 sq. ft. In another embodiment thiabendazole is applied to the upper surface of the substrate in an amount from 1-500 g per 100 m², preferably 2-450 g per 100 m², more preferably 5-400 g per 100 m² and in particular 10-300 g per 100 m². It is well known to a person skilled in the art that application volumes may differ depending on the concentration of thiabendazole in the compositions applied. Usually, diluted thiabendazole compositions are applied in a higher volume per surface area unit than concentrated thiabendazole compositions. It is well within the reach of the skilled artisan to calculate the effective amount of thiabendazole that needs to be applied to a certain surface area. Thiabendazole is for instance commercialised as a composition comprising 50 wt % thiabendazole. It is advised to apply 20 fl. oz. per 1000 sq. ft. of this composition to the upper surface of the substrate in the US, while it is advised to apply 8 fl. oz. per 1000 sq. ft. of this composition to the upper surface of the substrate in the Canada.

In a further aspect the invention relates to a method for producing mushrooms, the method comprising the steps of: a) providing a substrate wherein mushrooms are to be grown, b) inoculating the substrate with mushroom spawn, c) adding a casing layer to the substrate, d) applying natamycin and thiabendazole to the substrate, e) applying conditions to stimulate growth of the mushrooms, and f) harvesting the mushrooms. Any of the above-described features of the method for controlling a fungal disease in the production of mushrooms can also be applied in this method. In an embodiment natamycin and thiabendazole can also be applied to the substrate during step e. Natamycin and thiabendazole can also be applied to the substrate after step f, i.e. after a first harvest and before the new mushrooms move towards maturity.

A further aspect of the invention is directed to a product treated with natamycin and thiabendazole. The invention is therefore directed to a product comprising natamycin and thiabendazole. The treated products may comprise natamycin and thiabendazole on their surface and/or inside the product. In a preferred embodiment the product is an agricultural product including, but not limited to, a substrate wherein mushrooms are growing or are to be grown, a casing layer, mushroom spawn, a supplement, a mushroom.

A further aspect of the invention relates to the use of natamycin and thiabendazole for controlling a fungal disease during the production of mushrooms.

So, when the substrate (i.e. compost) wherein mushrooms are growing or are to be grown comprises natamycin and thiabendazole, these compounds can already be incorporated into the substrate during the phase 1 and/or phase 2 composting step.

When the mushroom spawn comprises the antifungal compounds, they can be incorporated into the substrate at the spawning step.

When the casing layer comprises the antifungal compounds, they can be incorporated into the substrate at the casing step. The compounds can be incorporated in the material used for casing and applied to the substrate when the casing layer is applied. This way the antifungal compounds are well dispersed throughout the casing layer. The compounds can be formulated in solid form or on solid carriers. Alternatively, the compounds can be sprayed onto the casing layer after it has been applied to the substrate.

When a supplement comprises the antifungal compounds, they can be incorporated into the substrate preferably at the composting step, the spawning step, and/or the casing step. Finally, when natamycin and thiabendazole are applied to a substrate, wherein mushrooms are grown, the matured mushrooms may comprise the compounds on their surface or the compounds may even be incorporated into the mushroom.

### EXAMPLES

### Example 1

Synergistic antifungal activity of combined application of natamycin and thiabendazole

To demonstrate synergistic antifungal activity of the combination of natamycin with thiabendazole against Trichoderma harzianum, an in vitro assay was conducted using 96-well microtiter plates. The following compositions are tested:
Control (no active ingredient),
1.25 ppm natamycin (DSM Food Specialties, Delft, The Netherlands),
0.5, 1.0, 1.25 or 1.5 ppm thiabendazole,
1.25 ppm natamycin+0.5 ppm thiabendazole,
1.25 ppm natamycin+1.0 ppm thiabendazole,
1.25 ppm natamycin+1.25 ppm thiabendazole,
1.25 ppm natamycin+1.5 ppm thiabendazole.
   After filling each well of a microtiter plate with 80 μl of PCB medium, the active ingredient(s) were added from separate stock solutions prepared in methanol, which resulted in an intermediate volume of 100 μl per well. Subsequently, 100 μl of a Trichoderma harzianum suspension prepared in PCB medium is used to inoculate each well with 5.0×10³ spores/ml. Each well thus contained a final volume of 200 μl and <1% of methanol, which did not affect growth of Trichoderma harzianum (data not shown).

After incubation of the microtiter plates for 3, 5, 10 and 17 days at 25° C., the in vitro antifungal activity (%) of the individual active ingredients was assessed by calculating the reduction in mould growth observed in the presence of the active ingredient in comparison to the mould growth observed in the absence of the active ingredient. The expected antifungal activity (E in %) of the active ingredient combination was calculated according to the Colby equation (Colby, 1967):

E=X+Y−[(X·Y)/100]

wherein X and Y are the observed antifungal activities (in %) of the individual active ingredients X and Y, respectively. If the observed antifungal activity (O in %) of the combination exceeds the expected antifungal activity (E in %) of the combination and the resulting synergy factor O/E is thus >1.0, the combined application of the active ingredients leads to a synergistic antifungal effect.

The results (see Table 1) demonstrate that the natamycin+thiabendazole combination has a much stronger antifungal activity against Trichoderma harzianum than natamycin or thiabendazole alone. The observed antifungal activity of the combination natamycin+thiabendazole was 50 to 100% higher than the expected antifungal activity and a synergy factor far above 1.0 was therefore obtained.

The results of this example clearly show that the combined application of natamycin and thiabendazole synergistically inhibit growth of Trichoderma harzianum. It is thus advantageous to use the combination of natamycin and thiabendazole to control green mold disease in mushrooms.

TABLE 1
In vitro antifungal activity (%) of natamycin in combination with
thiabendazole against Trichoderma harzianum after incubation at 25° C.

| | | Observed | Expected | |
|---|---|---|---|---|
| | Incubation | antifungal | antifungal | Synergy |
| | time | activity | activity | factor |
| Antifungal composition | (days) | O (%) | E (%) | O/E |
| Control | 3 | 0 | — | — |
| Natamycin 1.25 ppm | | 0 | — | — |
| Thiabendazole 0.5 ppm | | 0 | — | — |
| Natamycin 1.25 ppm + | | 100 | 0 | >100 |
| Thiabendazole 0.5 ppm | | | | |
| Control | 5 | 0 | — | — |
| Natamycin 1.25 ppm | | 0 | — | — |
| Thiabendazole 1.0 ppm | | 0 | — | — |
| Thiabendazole 1.25 ppm | | 0 | — | — |
| Natamycin 1.25 ppm + | | 50 | 0 | >50 |
| Thiabendazole 1.0 ppm | | | | |
| Natamycin 1.25 ppm + | | 100 | 0 | >100 |
| Thiabendazole 1.25 ppm | | | | |
| Control | 10 | 0 | — | — |
| Natamycin 1.25 ppm | | 0 | — | — |
| Thiabendazole 1.25 ppm | | 0 | — | — |
| Thiabendazole 1.5 ppm | | 0 | — | — |
| Natamycin 1.25 ppm + | | 50 | 0 | >50 |
| Thiabendazole 1.25 ppm | | | | |
| Natamycin 1.25 ppm + | | 100 | 0 | >100 |
| Thiabendazole 1.5 ppm | | | | |
| Control | 17 | 0 | — | — |
| Natamycin 1.25 ppm | | 0 | — | — |
| Thiabendazole 1.5 ppm | | 0 | — | — |
| Natamycin 1.25 ppm + | | 100 | 0 | >100 |
| Thiabendazole 1.5 ppm | | | | |

### REFERENCES

Colby SR (1967), Calculating synergistic and antagonistic responses of herbicide combination. Weeds 15:20-22.

Gea FJ, Tello JC and Honrubia M (1997), In vitro sensitivity of Verticillium fungicola to selected fungicides. Mycopathologia 136: 133-137.

Grogan H (2008) Challenges facing mushroom disease control in the 21^{st} century. Proceedings of the 6^{th} International Conference on Mushroom Biology and Mushroom Products 120-127.

Romaine CPD, Royse DJ and Schlagnhaufer C (2005), Superpathogenic Trichoderma resistant to TopsinM found in Pennsylvania and Delaware. Mushroom News 53:6-9.

Romaine CPD, Royse DJ and Schlagnhaufer C (2008), Emergence of benzimidazole-resistant green mold, Trichoderma aggresivum, on cultivated Agaricus bisporus in North America. Mush. Sci. 17:510-523.

Slinker BK (1998), The Statistics of Synergism. Journal of Mol. and Cell. Cardiology 30:723-731.

1. A method for controlling a fungal disease during production of mushrooms comprising applying natamycin and thiabendazole to a substrate wherein mushrooms are growing and/or are to be grown.

2. A method according to claim 1, wherein natamycin and thiabendazole are applied in a single composition.

3. A method according to claim 1, wherein the fungal disease is caused by a Verticillium species, a Mycogone species or a Trichoderma species.

4. A method according to claim 1, wherein natamycin and thiabendazole are applied to the substrate after spawning.

5. A method according to claim 1, wherein natamycin and thiabendazole are applied to the substrate after casing.

6. A method according to claim 1, wherein natamycin and thiabendazole are applied more than once during production of mushrooms.

7. A method according to claim 1, wherein natamycin and thiabendazole are applied by spraying.

8. A method according to claim 1, wherein natamycin is applied to an upper surface of the substrate in an amount from 0.1-500 g per 100 m².

9. A method according to claim 1, wherein thiabendazole is applied to an upper surface of the substrate in an amount from 1-500 g per 100 m².

10. A method for producing mushrooms, the method comprising:
a. providing a substrate wherein mushrooms are to be grown,
b. inoculating the substrate with mushroom spawn,
c. adding a casing layer to the substrate,
d. applying natamycin and thiabendazole to the substrate,
e. applying conditions to stimulate growth of the mushrooms, and
f. harvesting the mushrooms.

11. A method according to claim 10, wherein natamycin and thiabendazole are also applied to the substrate during e.

12. A substrate wherein mushrooms are growing or are to be grown, the substrate comprising natamycin and thiabendazole.

13. A casing layer comprising natamycin and thiabendazole.

14. A mushroom spawn comprising natamycin and thiabendazole.

15. A mushroom comprising natamycin and thiabendazole.
